(19) United States
(12) Patent Application Publication
(10) Pub. No.: US 2001/0049368 A1
(43) Pub. Date: Dec. 6, 2001

(54) Method for treating or preventing depression

(76) Inventors: Scott A. Reines, New Hope, PA (US); Annette Robichaud, Montreal (CA); Frederick D. Tattersall, Bishops Stortford (GB)

Correspondence Address: MERCK AND CO INC, P O BOX 2000, RAHWAY, NJ, 070650907

(21) Appl. No.: 09/794,197

(22) Filed: Feb. 27, 2001

Related U.S. Application Data
(60) Provisional application No. 60/185,632, filed on Feb. 29, 2000.

(51) Int. Cl. A61K 31/5377

(52) U.S. Cl. 514/235.8

(57) ABSTRACT

A neurokinin-1 antagonist or an alpha-2 adrenoreceptor agonist provide an effective therapy in conjunction with a PDE4 inhibitor for the treatment or prevention of depression and/or anxiety. These combinations minimize the side effects of nausea and/or emesis associated with the PDE4 inhibitor and may also provide beneficial antidepressant and/or anti-anxiety effects.

### BACKGROUND OF THE INVENTION

Depression is characterised by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions.

Treatment regimens commonly include the use of tricyclic antidepressants, monoamine oxidase inhibitors, some psychotropic drugs, lithium carbonate, and electroconvulsive therapy (ECT) (see R. J. Baldessarini in Goodman & Gilman's The Pharmacological Basis of Therapeutics, 9th Edition, Chapter 19, McGraw-Hill, 1996 for a review). More recently, new classes of antidepressant drugs are being developed including selective serotonin reuptake inhibitors (SSRIs), specific monoamine reuptake inhibitors and 5-HT1A receptor agonists, antagonists and partial agonists.

Anxiety is an emotional condition characterised by feelings such as apprehension and fear accompanied by physical sympoms such as tachycardia, increased respiration, sweating and tremor. It is a normal emotion but when it is severe and disabling it becomes pathological. Anxiety disorders are generally treated using benzodiazepine sedative-antianxiety agents. Potent benzodiazepines are effective in panic disorder as well as in generalised anxiety disorder, however, the risks associated with drug dependency may limit their long-term use. 5-HT1A receptor partial agonists also have useful anxiolytic and other psychotropic activity, and less likelihood of sedation and dependance (see R. J. Baldessarini in Goodman & Gilman's The Pharmacological Basis of Therapeutics, 9th Edition, Chapter 18, McGraw-Hill, 1996 for a review).

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′,5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of compounds for the treatment or prevention of depression and/or anxiety. PDE4 is known to exist as at lease four isoenzymes, each of which is encoded by a distinct gene. Each of the four known PDE4 gene products is believed to play varying roles in allergic and/or inflammatory responses. Thus, it is believed that inhibition of PDE4, particularly the specific PDE4 isoforms that produce detrimental responses, can be utilized for the treatment or prevention of depression and/or anxiety.

Inhibition of type IV cyclic nucleotide phosphodiesterase (PDE) has been investigated as a novel approach to the treatment of depression. (Zeller E., et al., Pharmacopsychiatria 17: 188-190 (1984); Horowski R., et al., Current Therapeutic Res. 38: 23-29 (1985)).

A major concern with the use of PDE4 inhibitors is the side effect of emisis which has been observed for several candidate compounds as described in C. Burnouf et al., (“Burnouf”), Ann. Rep. In Med. Chem., 33:91-109(1998). B. Hughes et al., Br. J. Pharmacol., 118:1183-1191(1996); M. J. Perry et al., Cell Biochem. Biophys., 29:113-132(1998); S. B. Christensen et al., J. Med. Chem., 41:821-835(1998); and Burnouf describe the wide variation of the severity of the undesirable side effects exhibited by various compounds. As noted in M. D. Houslay et al., Adv. In Pharmacol., 44:225-342(1998) and D. Spina et al., Adv. In Pharmacol., 44:33-89(1998), there is great interest and research of therapeutic PDE4 inhibitors. However, the side effects associated with PDE4 inhibitors would limit their use in the treatment or prevention of depression and/or anxiety.

The neuropeptide receptors for substance P (neurokinin-1; NK-1) are widely distributed throughout the mammalian nervous system (especially brain and spinal ganglia), the circulatory system and peripheral tissues (especially the duodenum and jejunum) and are involved in regulating a number of diverse biological processes. This includes sensory perception of olfaction, vision, audition and pain, movement control, gastric motility, vasodilation, salivation, and micturition (B. Pernow, Pharmacol. Rev., 1983, 35, 85-141).

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to SP the known mammalian tachykinins include neurokinin A and neurokinin B. The current nomenclature designates the receptors for substance P, neurokinin A, and neurokinin B as neurokinin-1, neurokinin-2, and neurokinin-3, respectively.

Neurokinin 1 (NK-1; substance P) receptor antagonists are being developed for the treatment of a number of physiological disorders associated with an excess or imbalance of tachykinins, and in particular substance P. Examples of such conditions include disorders of the central nervous system such as anxiety, depression and psychosis.

Activation of alpha2-adrenoceptors with agonists such as clonidine or xylazine has been shown to elicit dose-dependent vomiting in cats and dogs (Hikasa et al., Am. J. Vet. Res., 50, 1348-1351 (1989); Hikasa et al., J. Pharmacol. Exp. Therap., 261, 746-754 (1992); Hikasa et al., Eur. J. Pharmacol., 229, 241-251 (1992); Japundzic-Zigon et al., Pharmacol. Res., 35, 287-297 (1997)). In these species, emesis induced by clonidine or xylazine is thought to be mediated by alpha₂-adrenoceptors, since it is prevented by drugs with alpha₂-adrenoceptor antagonistic activity, such as yohimbine (Hikasa et al., Am. J. Vet. Res., 50, 1348-1351 (1989); Hikasa et al., J. Pharmacol. Exp. Therap., 261, 746-754 (1992); Hikasa et al., Eur. J. Pharmacol., 229, 241-251 (1992)). Further therapeutic indications of alpha-2 adrenoceptor agonists have been discussed in the literature (Ruffolo, et al, Annual Review of Pharmacology & Toxicology, 32, 243-279 (1993)).

There remains a need in the art for an effective therapy for the treatment or prevention of depression and/or anxiety which utilizes a PDE4 inhibitor yet minimizes the side effect of nausea and/or emesis associated with the PDE4 inhibitor. Surprisingly, a neurokinin-1 antagonist or an alpha-2 adrenoreceptor agonist provide an effective therapy in conjunction with a PDE4 inhibitor for the treatment or prevention of depression and/or anxiety. Such combinations exhibit unexpected and advantageous results, for example, by minimizing the side effects of nausea and/or emesis associated with the PDE4 inhibitor and, optionally, providing additional antidepressant and/or anti-anxiety effect.

Although the present invention is not limited to a specific mechanism of action, the inventors postulate that a neurokinin-1 antagonist and/or an alpha-2 adrenoreceptor agonist would be effective in conjunction with a PDE4 inhibitor for the treatment or prevention of depression and/or anxiety while minimizing the side effect of nausea and/or emesis associated with the PDE4 inhibitor. While not being bound to any particular theory of operation, an enhanced effect at treating or preventing a psychological stress response in an animal assay is observed with the combination of drugs than would be expected from either drug alone. In particular, combination therapy of a neurokinin-1 antagonist or an alpha-2 adrenoreceptor agonist and a PDE4 inhibitor provide an effective therapy for the treatment or prevention of depression and/or anxiety.

### SUMMARY OF THE INVENTION

The present invention relates to the treatment or prevention of depression and/or anxiety by the administration of a PDE4 inhibitor in conjunction with a neurokinin-1 antagonist and/or an alpha-2 adrenoreceptor agonist. In particular, the use of a PDE4 inhibitor in conjunction with a neurokinin-1 antagonist or an alpha-2 adrenoreceptor agonist provides an effective therapy a for the treatment or prevention of depression and/or anxiety. These combinations minimize the side effects of nausea and/or emesis associated with the PDE4 inhibitor and may also provide beneficial antidepressant and/or anti-anxiety effects. a combination of a specific class of NK-1 receptor antagonists and an antidepressant or anti-anxiety agent.

### DESCRIPTION OF THE INVENTION

The present invention relates to methods for the treatment or prevention of depression and/or anxiety by administering a PDE4 inhibitor in conjunction with a neurokinin-1 antagonist or an alpha-2 adrenoreceptor agonist. The present methods for the treatment or prevention of depression in a patient which comprises administering the combination of a PDE4 inhibitor and a neurokinin-1 receptor antagonist and/or an alpha-2 adrenoreceptor agonist, such that they give effective relief.

The present invention further relates to a method for the treatment or prevention of depression in a patient which comprises administering the combination of a PDE4 inhibitor and a neurokinin-1 receptor antagonist or an alpha-2 adrenoreceptor agonist, wherein the neurokinin-1 receptor antagonist or the alpha-2 adrenoreceptor agonist minimizes the side effects of nausea and/or emesis associated with the PDE4 inhibitor.

The present invention accordingly provides the use of a PDE4 inhibitor and a neurokinin-1 receptor antagonist and/or an alpha-2 adrenoreceptor agonist for the manufacture of a medicament for the treatment or prevention of depression and/or anxiety.

The present invention also provides a method for the treatment or prevention of depression and/or anxiety, which method comprises administration to a patient in need of such treatment an amount of a PDE4 inhibitor and a neurokinin-1 receptor antagonist and/or an alpha-2 adrenoreceptor agonist, such that together they give effective relief.

In a further aspect of the present invention, there is provided a pharmaceutical composition comprising a PDE4 inhibitor and a neurokinin-1 receptor antagonist and/or an alpha-2 adrenoreceptor agonist, together with at least one pharmaceutically acceptable carrier or excipient.

It will be appreciated that the PDE4 inhibitor and the neurokinin-1 receptor antagonist and/or the alpha-2 adrenoreceptor agonist may be present as a combined preparation for simultaneous, separate or sequential use for the treatment or prevention of depression and/or anxiety. Such combined preparations may be, for example, in the form of a twin pack.

In a further or alternative aspect of the present invention, there is therefore provided a product comprising a PDE4 inhibitor and a neurokinin-1 receptor antagonist and/or an alpha-2 adrenoreceptor agonist as a combined preparation for simultaneous, separate or sequential use in the treatment or prevention of depression and/or anxiety.

The present invention is further directed to a method for ameliorating the symptoms attendant to depression and/or anxiety in a patient comprising the administration of a PDE4 inhibitor and a neurokinin-1 receptor antagonist and/or an alpha-2 adrenoreceptor agonist.

The present invention is further directed to a method for ameliorating the symptoms attendant to the use of a PDE4 inhibitor for the treatment or prevention of depression and/or anxiety in a patient comprising administering to the patient a neurokinin-1 receptor antagonist and/or an alpha-2 adrenoreceptor agonist.

The present invention is further directed to a method for reducing nausea and/or emesis associated with employing a PDE4 inhibitor for the treatment or prevention of depression and/or anxiety in a patient comprising administering to the patient a neurokinin-1 receptor antagonist and/or an alpha-2 adrenoreceptor agonist.

In accordance with the present invention the PDE4 inhibitor and the neurokinin-1 receptor antagonist and/or the alpha-2 adrenoreceptor agonist are administered to a patient in a quantity sufficient to treat or prevent the symptoms and/or underlying etiology associated with depression and/or anxiety in a patient.

The present invention also provides the use of a PDE4 inhibitor and a neurokinin-1 receptor antagonist and/or an alpha-2 adrenoreceptor agonist for the manufacture of a medicament for treating or preventing depression and/or anxiety in a patient.

Although the present invention is useful in any mammal suffering from depression and/or anxiety a preferred subject is a human. Although the present invention is applicable to humans of any age, it may find greater application in elderly patients.

It will be appreciated that when using a combination of the present invention, the PDE4 inhibitor and the neurokinin-1 receptor antagonist and/or the alpha-2 adrenoreceptor agonist will be administered to a patient, within a reasonable period of time. The compounds may be in a unitary formulation of pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially, concurrently or concombinantly. Therefore, by way of example, the PDE4 inhibitor may be administered as a tablet and then, within a reasonable period of time, the neurokinin-1 receptor antagonist or the alpha-2 adrenoreceptor agonist may be administered as an oral dosage form such as a tablet, or another dosage form which provides effective delivery of the medicament.

By “reasonable period of time” is meant a time period that is such that the therapeutic efficacy of the compounds overlap. That is, for example, if the PDE4 inhibitor is provided as a tablet, then within the PDE4 inhibitor's window of therapeutic efficacy, the neurokinin-1 receptor antagonist or the alpha-2 adrenoreceptor agonist should be administered, either in the same type of dosage form, or another dosage form which provides effective delivery of the medicament.

The compositions of the present invention are useful for the treatment of depression. As used herein, the term “depression” includes depressive disorders, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder.

Other mood disorders encompassed within the term “depression” include dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

The compositions of the present invention are useful for the treatment of anxiety. As used herein, the term “anxiety” includes anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

“Generalised anxiety” is typically defined as an extended period (e.g. at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep.

“Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterised by an anxiety about being in places or situations from which escape might be difficult or embarrassing or in which help may not be available in the event of a panic attack. Agoraphobia may occur without history of a panic attack. A “specific phobia” is characterised by clinically significant anxiety provoked by exposure to a specific feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterised by clinically significant anxiety provoked by exposure to certain types of social or performance circumstances. Social phobia may also be referred to as social anxiety disorder.

Other anxiety disorders encompassed within the term “anxiety” include anxiety disorders induced by alcohol, amphetamines, caffeine, cannabis, cocaine, hallucinogens, inhalants, phencyclidine, sedatives, hypnotics, anxiolytics and other substances, and adjustment disorders with anxiety or with mixed anxiety and depression.

Anxiety may be present with or without other disorders such as depression in mixed anxiety and depressive disorders. The compositions of the present invention are therefore useful in the treatment of anxiety with or without accompanying depression.

The combinations of the present invention are especially useful for the treatment of or prevention of depression and/or anxiety where the use of another antidepressant or anti-anxiety agent is generally prescribed. By the use of a combination of a PDE4 inhibitor and a neurokinin-1 receptor antagonist and/or an alpha-2 adrenoreceptor agonist in accordance with the present invention, it is now also possible to treat or prevent depression and/or anxiety in patients for whom conventional antidepressant or anti-anxiety therapy might not be wholly successful or where dependance upon the antidepressant or anti-anxiety therapy is prevalent.

The combinations of the present invention are also useful for the treatment of or prevention of depression and/or anxiety in conjunction with the use of other antidepressant or anti-anxiety agents known in the art.

Suitable classes of other antidepressant agents of use in conjunction with the present invention include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists and atypical antidepressants.

Another class of antidepressant agents of use in conjunction with the present invention are noradrenergic and specific serotonergic antidepressants (NaSSAs). A suitable example of a NaSSA is mirtazapine. Suitable norepinephrine reuptake inhibitors of use conjunction with in the present invention include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof. Another norepinephrine reuptake inhibitor of use in conjunction with the present invention is reboxetine. Suitable selective serotonin reuptake inhibitors of use in conjunction with the present invention include: fluoxetine, fluvoxamine, paroxetine and sertraline, and pharmaceutically acceptable salts thereof. Suitable monoamine oxidase inhibitors of use in conjunction with the present invention include: isocarboxazid, phenelzine, tranylcypromine and selegiline, and pharmaceutically acceptable salts thereof. Suitable reversible inhibitors of monoamine oxidase of use in conjunction with the present invention include: moclobemide, and pharmaceutically acceptable salts thereof. Suitable serotonin and noradrenaline reuptake inhibitors of use in conjunction with the present invention include: venlafaxine, and pharmaceutically acceptable salts thereof. Suitable CRF antagonists of use in conjunction with the present invention include those compounds described in International Patent Specification Nos. WO 94/13643, WO 94/13644, WO 94/13661, WO 94/13676 and WO 94/13677. Suitable atypical antidepressants of use in conjunction with the present invention include: bupropion, lithium, nefazodone, trazodone and viloxazine, and pharmaceutically acceptable salts thereof. Another suitable atypical antidepressant is sibutramine. Other antidepressants of use in conjunction with the present invention include adinazolam, alaproclate, amineptine, amitriptyline/chlordiazepoxide combination, atipamezole, azamianserin, bazinaprine, befuraline, bifemelane, binodaline, bipenamol, brofaromine bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, montirelin, nebracetam, nefopam, nialamide, nomifensine, norfluoxetine, orotirelin, oxaflozane, pinazepam, pirlindone, pizotyline, ritanserin, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometapine, and pharmaceutically acceptable salts thereof, and St. John's wort herb, or Hypericum perforatum, or extracts thereof.

Suitable classes of anti-anxiety agent of use in conjunction with the present invention include benzodiazepines and 5-HT_{1A} agonists or antagonists, especially 5-HT_{1A} partial agonists, and corticotropin releasing factor (CRF) antagonists. In addition to benzodiazepines, other suitable classes of anti-anxiety agent are nonbenzodiazepine sedative-hypnotic drugs such as zolpidem; mood-stabilizing drugs such as clobazam, gabapentin, lamotrigine, loreclezole, oxcarbamazepine, stiripentol and vigabatrin; and barbiturates.

Suitable benzodiazepines of use in conjunction with the present invention include: alprazolam, chlordiazepoxide, clonazepam, chlorazepate, diazepam, halazepam, lorazepam, oxazepam and prazepam, and pharmaceutically acceptable salts thereof. Suitable 5-HT_{1A} receptor agonists or antagonists of use in conjunction with the present invention include, in particular, the 5-HT_{1A} receptor partial agonists buspirone, flesinoxan, gepirone and ipsapirone, and pharmaceutically acceptable salts thereof. An example of a compound with 5-HT_{1A} receptor antagonist/partial agonist activity is pindolol. Suitable CRF antagonists of use in conjunction with the present invention include those compounds described in International Patent Specification Nos. WO 94/13643, WO 94/13644, WO 94/13661, WO 94113676 and WO 94/13677. Another class of anti-anxiety agent of use in conjunction with the present invention are compounds having muscarinic cholinergic activity. Suitable compounds in this class include ml muscarinic cholinergic receptor agonists such as those compounds described in European Patent Specification Nos. 0 709 093, 0 709 094 and 0 773 021, and International patent Specification No. WO 96/12711. Another class of anti-anxiety agent of use in conjunction with the present invention are compounds acting on ion channels. Suitable compounds in this class include carbamazepine, lamotrigine and valproate, and pharmaceutically acceptable salts thereof.

The PDE4 inhibitors, the neurokinin-1 receptor antagonists and the alpha-2 adrenoreceptor agonists of use in the present invention may be any PDE4 inhibitor, neurokinin-1 receptor antagonist or alpha-2 adrenoreceptor agonist known from the art.

The PDE4 inhibitor may be peptidal or non-peptidal in nature, however, the use of a non-peptidal PDE4 inhibitor is preferred. In a preferred embodiment, the PDE4 inhibitor is a CNS-penetrant PDE4 inhibitor. In addition, for convenience the use of an orally active PDE4 inhibitor is preferred. To facilitate dosing, it is also preferred that the PDE4 inhibitor is a long acting PDE4 inhibitor. An especially preferred class of PDE4 inhibitors of use in the present invention are those compounds which are orally active and long acting.

Representative PDE4 inhibitors of use in the present invention are fully described, for example, in U.S. Pat. Nos. 5,340,827, 5,550,137, 5,491,147, 5,608,070, 5,622,977, 5,633,257, 5,739,144, 5,776,958, 5,780,477, 5,780,478, 5,786,354, 5,798,373, 5,580,888, 5,849,770, 5,859,034, 5,866,593, 5,891,896, 5,919,801, 6,005,118 and International Patent Publications WO 94/22852, WO 95/35283, WO 96/00215. Such PDE4 inhibitors include pentoxifylline, isobutylmethylxanthine, ciprofloxacin, rolipram and terferol. In a preferred embodiment of the present invention, the PDE4 inhibitor is a compound other than rolipram.

The alpha-2 adrenoreceptor agonist may be peptidal or non-peptidal in nature, however, the use of a non-peptidal alpha-2 adrenoreceptor agonist is preferred. In a preferred embodiment, thealpha-2 adrenoreceptor agonist is a CNS-penetrant alpha-2 adrenoreceptor agonist. In addition, for convenience the use of an orally active alpha-2 adrenoreceptor agonist is preferred. To facilitate dosing, it is also preferred that the alpha-2 adrenoreceptor agonist is a long acting alpha-2 adrenoreceptor agonist. An especially preferred class of alpha-2 adrenoreceptor agonists of use in the present invention are those compounds which are orally active and long acting.

Alpha-2 adrenoreceptor agonists of use in the present invention are fully described, for example, in U.S. Pat. Nos. 4,454,139, 4,473,572, 4,481,200, 4,550,114, 4,604,398, 4,640,924, 4,717,731, 5,914,342, 4,923,865, 5,804,587, 5,916,900 and 5,965,595.

Specific alpha-2 adrenoreceptor agonists of use in the present invention include clonidine, labetalol, para-aminoclonidine and xylazine.

The neurokinin-1 receptor antagonist may be peptidal or non-peptidal in nature, however, the use of a non-peptidal neurokinin-1 receptor receptor antagonist is preferred. In a preferred embodiment, the neurokinin-1 receptor antagonist is a CNS-penetrant neurokinin-1 receptor antagonist. In addition, for convenience the use of an orally active neurokinin-1 receptor receptor antagonist is preferred. To facilitate dosing, it is also preferred that the neurokinin-1 receptor antagonist is a long acting neurokinin-1 receptor antagonist. An especially preferred class of neurokinin-1 receptor antagonists of use in the present invention are those compounds which are orally active and long acting.

Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434,0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689.

Specific neurokinin-1 receptor antagonists of use in the present invention include:

(±)-(2R3R, 2S3S)-N-{[2-cyclopropoxy-5-(trifluoromethoxy)-phenyl]methyl}-2-phenylpiperidin-3-amine;

2-(S)-(3,5-bis(trifluoromethyl)benzyloxy)-3(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;

2-(S)-(3,5-bis(trifluoromethyl)benzyloxy)-4-(3-(5-oxo-1H,4H-1,2,4-triazolo)methyl)-3-(S)-phenyl-morpholine;

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;

(3S,5R,6S)-3-[2-cyclopropoxy-5-(trifluoromethoxy)phenyl] -6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;

2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

2-(R)-(1-(R)-(3,5-bi s(trifluoromethyl)phenyl)ethoxy)-3-(S)-(4-fluorophenyl)-4-(3-(1-monophosphoryl-5-oxo-1H-1,2,4-triazolo)methyl)morpholine;

2-(R)-(1-(R)-(3,5-bi s(trifluoromethyl)phenyl)ethoxy)-3-(S)-(4-fluorophenyl)-4-(3-(2-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;

2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

or a pharmaceutically acceptable salt thereof.

The preparation of the such compounds is fully described in the aforementioned patents and publications.

Suitable pharmaceutically acceptable salts of the compounds of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

The above compounds are only illustrative of the PDE4 inhibitors, neurokinin-1 receptor antagonists and alpha-2 adrenoreceptor agonists which are currently under investigation. As this listing of compounds is not meant to be comprehensive, the methods of the present invention may employ any PDE4 inhibitor, neurokinin-1 receptor antagonist or alpha-2 adrenoreceptor agonist. Accordingly, the present invention is not strictly limited to any particular structural class of compound.

The identification of a compound as a PDE4 inhibitor, a neurokinin-1 receptor antagonist or an alpha-2 adrenoreceptor agonist, and thus able to have utility in the present invention may be readily determined without undue experimentation by methodology well known in the art.

The PDE4 inhibitor, neurokinin-1 receptor antagonist and the alpha-2 adrenoreceptor agonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Preferably the compositions according to the present invention are in unit dosage forms such as tablets, pills, capsules, powders, granules, solutions or suspensions, or suppositories, for oral, parenteral or rectal administration, by inhalation or insulation or administration by trans-dermal patches or by buccal cavity absorption wafers.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Preferred compositions for administration by injection include those comprising a PDE4 inhibitor, a neurokinin-1 receptor antagonist or an alpha-2 adrenoreceptor agonist as the active ingredient, in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g. Tween 20, 40, 60, 80 or 85) and other sorbitans (e.g. Span 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid, Liposyn, Infonutrol, Lipofundin and Lipiphysan. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 cm, particularly 0.1 and 0.5 μm, and have a pH in the range of 5.5 to 8.0.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Compositions of the present invention may also be presented for administration in the form of trans-dermal patches using conventional technology. The compositions may also be administered via the buccal cavity using, for example, absorption wafers.

Compositions in the form of tablets, pills, capsules or wafers for oral administration are particularly preferred.

In the present invention, the PDE4 inhibitor and the neurokinin-1 receptor antagonist or the alpha-2 adrenoreceptor agonist may be independently present in dose ranges from one one-hundredth to one times the dose levels which are effective when these compounds are used singly. In combination therapy of the present invention, the PDE4 inhibitor may be administered with the neurokinin-1 receptor antagonist or the alpha-2 adrenoreceptor agonist (e.g., concurrently, concombinantly, sequentially, or in a unitary formulation) such that their therapeutic efficacy overlap.

Typically, the individual daily dosages for these combinations may range from about one-fifth of the minimally recommended clinical dosages to the maximum recommended levels for the entities when they are given singly.

To illustrate these combinations, a PDE4 inhibitor effective clinically at a given daily dose range may be effectively combined, at levels which are equal or less than the daily dose range, with the neurokinin-1 receptor antagonist or the alpha-2 adrenoreceptor agonist.

Naturally, these dose ranges may be adjusted on a unit basis as necessary to permit divided daily dosage and, as noted above, the dose will vary depending on the nature and severity of the disease, weight of patient, special diets and other factors. These combinations may be formulated into pharmaceutical compositions as known in the art and as discussed herein.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease or disorder, the patient's weight, special diets then being followed by a patient, concurrent medication, the intrinsic tachykinin receptor antagonist activity of the compound, the bioavailability upon oral administration of the compound and other factors which those skilled in the art will recognize.

In the treatment of a condition in accordance with the present invention, an appropriate dosage level will generally be about 0.01 μg to 50 mg per kg patient body weight per day which may be administered in single or multiple doses. Preferably, the dosage level will be about 0.1 μg to about 25 mg/kg per day; more preferably about 0.5 μg to about 10 mg/kg per day. For example, for treating or preventing depression and/or anxiety or ameliorating the symptoms attendant to depression and/or anxiety in a patient, a suitable dosage level is about 0.1 μg to 25 mg/kg per day, preferably about 0.5 μg to 10 mg/kg per day, and especially about 1 μg to 5 mg/kg per day. In larger mammals, for example humans, a typical indicated dose is about 300 μg to 400 mg orally. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day. In larger mammals, for example humans, a typical indicated dose is about 100 μg to 100 mg i.v. A compound may be administered on a regimen of several times per day, for example 1 to 4 times per day, preferably once or twice per day, and more preferably once a day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 300 mg active ingredient. A minimum dosage level for the NK-1 receptor antagonist is generally about 5 mg per day, preferably about 10 mg per day and especially about 20 mg per day. A maximum dosage level for the NK-1 receptor antagonist is generally about 1500 mg per day, preferably about 1000 mg per day and especially about 500 mg per day.

It will be appreciated that the amount of the PDE4 inhibitor and the neurokinin-1 receptor antagonist or the alpha-2 adrenoreceptor agonist in a patient will vary not only with the particular compounds or compositions selected but also with the route of administration, the nature of the condition being treated, and the age and condition of the patient, and will ultimately be at the discretion of the patient's physician or pharmacist. The length of time during which a tachykinin receptor antagonist will be given varies on an individual basis.

The present invention accordingly provides the use of a PDE4 inhibitor and a neurokinin-1 receptor antagonist and an alpha-2 adrenoreceptor agonist for the manufacture of a medicament adapted for oral administration for treating or preventing depression and/or anxiety in a patient.

The present invention also provides a method for treating or preventing depression and/or anxiety or ameliorating the symptoms attendant to depression and/or anxiety in a patient, which method comprises the oral administration to a patient in need of such treatment of an effective amount of an a PDE4 inhibitor and a neurokinin-1 receptor antagonist or an alpha-2 adrenoreceptor agonist.

In a further aspect of the present invention, there is provided an oral pharmaceutical composition for treating or preventing depression and/or anxiety in a patient, which comprises a PDE4 inhibitor and a neurokinin-1 receptor antagonist or an alpha-2 adrenoreceptor agonist, together with a pharmaceutically acceptable carrier or excipient.

It will be appreciated to those skilled in the art that reference herein to treatment extends to prophylaxis (prevention) as well as the treatment of the noted diseases/disorders and symptoms. Because the specific diagnosis of depression and/or anxiety in a particular patient may be difficult, the patient may benefit from the prophylactic administration of a subject compound in accordance with the present invention.

The following examples are provided for the purpose of further illustration only and are not intended to be limitations on the disclosed invention.

### EXAMPLE 1

PDE: LPS and fMLP-Induced TNF-α and LTB₄ Assays in Human Whole Blood

Whole blood provides a protein and cell-rich milieu appropriate for the study of biochemical efficacy of anti-inflammatory compounds such as PDE4-selective inhibitors. Normal non-stimulated human blood does not contain detectable levels of TNF-α and LTB₄. Upon stimulation with LPS, activated monocytes express and secrete TNF-α up to 8 hours and plasma levels remain stable for 24 hours. Published studies have shown that inhibition of TNF-α by increasing intracellular cAMP via PDE4 inhibition and/or enhanced adenylyl cyclase activity occurs at the transcriptional level. LTB₄ synthesis is also sensitive to levels of intracellular cAMP and can be completely inhibited by PDE4-selective inhibitors. As there is little LTB₄ produced during a 24 hour LPS stimulation of whole blood, an additional LPS stimulation followed by fMLP challenge of human whole blood is necessary for LTB4 synthesis by activated neutrophils. Thus, by using the same blood sample, it is possible to evaluate the potency of a compound on two surrogate markers of PDE4 activity in the whole blood by the following procedure.

Fresh blood was collected in heparinized tubes by venipuncture from healthy human volunteers (male and female). These subjects had no apparent inflammatory conditions and had not taken any NSAIDs for at least 4 days prior to blood collection. 500 μL aliquots of blood were pre-incubated with either 2μL of vehicle (DMSO) or 2 μL of test compound at varying concentrations for 15 minutes at 37° C. This was followed by the addition of either 10 μL vehicle (PBS) as blanks or 10 μL LPS (1 μg/mL final concentration, #L-2630 (Sigma Chemical Co., St. Louis, Mo.) from E. coli, serotype 0111 :B4; diluted in 0.1% w/v BSA (in PBS)). After 24 hours of incubation at 37° C., another 10 μL of PBS (blank) or 10 μL of LPS (1 μg/mL final concentration) was added to blood and incubated for 30 minutes at 37° C. The blood was then challenged with either 10 μL of PBS (blank) or 10 μL of fMLP (1 μM final concentration, #F-3506 (Sigma); diluted in 1% w/v BSA (in PBS)) for 15 minutes at 37° C. The blood samples were centrifuged at 1500×g for 10 minutes at 4° C. to obtain plasma. A 50 μL aliquot of plasma was mixed with 200 μL methanol for protein precipitation and centrifuged as above. The supernatant was assayed for LTB₄ using an enzyme immunoassay kit (#520111 from Cayman Chemical Co., Ann Arbor, MI) according to the manufacturer's procedure. TNF-α was assayed in diluted plasma (in PBS) using an ELISA kit (Cistron Biotechnology, Pine Brook, N.J.) according to manufacturer's procedure.

### EXAMPLE 2

SPA Based PDE Activity Assay Protocol

Compounds which inhibit the hydrolysis of cAMP to AMP by the type-IV cAMP-specific phosphodiesterases are screened in a 96-well plate format as follows: In a 96 well-plate at 30° C. was added the test compound (dissolved in 2 μL DMSO), 188 mL of substrate buffer containing [2,8-³H] adenosine 3′,5′-cyclic phosphate (cAMP, 100 nM to 50 μM), 10 mM MgCl₂, 1 mM EDTA, 50 mM Tris, pH 7.5. The reaction was initiated by the addition of 10 mL of human recombinant PDE4 (the amount was controlled so that ˜10% product was formed in 10min.). The reaction was stopped after 10 min. by the addition of 1 mg of PDE-SPA beads (Amersham Pharmacia Biotech, Inc., Piscataway, N.J.). The product AMP generated was quantified on a Wallac Microbeta® 96-well plate counter (EG&G Wallac Co., Gaithersburg, Md.). The signal in the absence of enzyme was defined as the background. 100% activity was defined as the signal detected in the presence of enzyme and DMSO with the background subtracted. Percentage of inhibition was calculated accordingly. IC₅₀ value was approximated with a non-linear regression fit using the standard 4-parameter/multiple binding sites equation from a ten point titration. The IC₅₀ values of PDE4 inhibitors are determined with 100 nM cAMP using the purified GST fusion protein of the human recombinant phosphodiesterase IVa (met-248) produced from a baculovirus/Sf-9 expression system.

### EXAMPLE 3

NK-1 Receptor Binding Assay

NK-1 receptor binding assays are performed in intact Chinese hamster ovary (CHO) cells expressing the human NK-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance P (0.1 nM, 2000 Ci/mmol; New England Nuclear) is incubated in the presence or absence of test compounds (dissolved in 5 μl dimethylsulphoxide, DMSO) with 5×10⁴ CHO cells. Ligand binding is performed in 0.25 ml of 50 mM Tris-HCl, pH7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2 μg/ml pepstatin, 2 μg/ml leupeptin and 2.8 μg/ml furoyl saccharine. The incubation proceeds at room temperature until equilibrium is achieved (>40 minutes) and the receptor-ligand complex is harvested by filtration over GF/C filters pre-soaked in 0.1% polyethylenimine using a Tomtek 96-well harvester. Non-specific binding is determined using excess substance P (1 μM) and represents <10% of total binding.

Particularly preferred NK-1 receptor antagonists of use in the present invention are compounds which are potent NK-1 receptor antagonists, i.e. compounds with an NK-1 receptor affinity (IC₅₀) of less than 10 nM, favourably less than 2 nM and preferably less than 1 nM.

Pharmacological assays for the study of antidepressant or anti-anxiety activity are well known in the art. Many are based upon the ability of antidepressants to support animal behaviour in stressful situations that ordinarily lead to diminished behavioural responsiveness (“learned helplessness”), such as repeated noxious shocks, forced swimming, or separation from other animals. For example, the following assay, which involves the inhibition of separation-induced vocalisations in guinea-pig pups, may be used to evaluate the methods of the present invention in the treatment or prevention of depression and/or anxiety.

### EXAMPLE 4

Separation-Induced Vocalisation

Male and female guinea-pigs pups are housed in family groups with their mothers and littermates throughout the study. Experiments are commenced after weaning when the pups are at least 2 weeks old. Before entering an experiment, the pups may be screened to ensure that a vigorous vocalisation response is reproducibly elicited following maternal separation. The pups are placed individually in an observation cage (approximately 55 cm×39 cm×19 cm) in a room physically isolated from the home cage for approximately 15 minutes and the duration and/or number of vocalisation during this baseline period is recorded. Those animals which vocalise for longer than 5 minutes may be employed for drug challenge studies (approximately 50% of available pups may fail to reach this criterion). On test days each pup receives an oral dose or an s.c. or i.p. injection of test compound or vehicle and is then immediately returned to the home cage with its mother and siblings, typically for at least 30 to 60 minutes (or for up to 4 hours following an oral dose, dependent upon the oral pharmacokinetics of the test compound) before social isolation for 15 minutes as described above. The duration and/or number of vocalisation on drug treatment days may be expressed as a percentage of the pre-treatment baseline value for each animal or compared with values obtained in vehicle-treated animals. The same subjects may be retested once weekly for up to 6 weeks. Between 6 and 8 animals typically receive each test compound at each dose tested.

### EXAMPLE 5

Gerbil Foot-Tapping (CNS Penetration) Assay

CNS-penetrant NK-1 receptor antagonists for use in the present invention can be identified by their ability to inhibit foot tapping in gerbils induced by anxiogenic agents (such as pentagastrin) or central infusion of NK-1 receptor agonists such as GR73632, or caused by aversive stimulation such as foot shock or single housing, based on the method of Rupniak & Williams, Eur. J. Pharmacol., 1994, 265, 179. Male or female Mongolian gerbils (35-70 g) are anaesthetised by inhalation of an isoflurane/oxygen mixture to permit exposure of the jugular vein in order to permit administration of test compounds or vehicle in an injection volume of approximately 5 ml/kg i.v. Alternatively, test compounds may be administered orally or by subcutaneous or intraperitoneal routes. A skin incision is then made in the midline of the scalp to expose the skull. An anxiogenic agent (e.g. pentagastrin) or a selective NK-1 receptor agonist (e.g. GR73632 (d Ala[L-Pro⁹,Me-Leu¹⁰]-substance P-(7-11)) is infused directly into the cerebral ventricles (e.g. 3 pmol in 5 μl i.c.v., depending on test substance) by vertical insertion of a cuffed 27 gauge needle to a depth of 4.5 mm below bregma. The scalp incision is closed and the animal allowed to recover from anaesthesia in a clear perspex observation box (approximately 25 cm×20 cm×20cm). The duration and/or intensity of hind foot tapping is then recorded continuously for approximately 5 minutes. Alternatively, the ability of test compounds to inhibit foot tapping evoked by aversive stimulation, such as foot shock or single housing, may be studied using a similar method of quantification.

It will be appreciated that CNS-penetration as defined by this assay and as used herein is a property of the NK-1 receptor antagonist and is not conferred by co-administration or co-formulation of the NK-1 receptor antagonist with a carrier or excipient designed to transiently open the blood-brain barrier.

One example of a NK-1 receptor antagonist active in the preclinical screens of the present invention is the compound 2-(R)-(1-(R)-(3,5-bis(trifluoro-methyl)phenyl)ethoxy)-3-(S)-(4-fluorophenyl)-4-(3-(5-oxo-1H,4H-1,2,4-triazolo)-methyl)morpholine, the preparation of which is described in International Patent Specification No. WO 95/16679. In the aforementioned assays, this compound has the following activity:

| | |
|---|---|
| human NK-1 receptor binding: | IC50 = 0.1 nM |
| guinea-pig vocalisation | ID50 = 0.73 mg/kg p.o. |
| (4 hrs. pretreatment) | |
| gerbil foot-tapping (5 mins.): | ID50 = 0.36 mg/kg i.v. |
| gerbil foot-tapping (24 hrs.): | ID50 = 0.33 mg/kg i.v. |

Another example of a NK-1 receptor antagonist active in the preclinical screens of the present invention is the compound 2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine, the preparation of which is described in International Patent Specification No. WO 95/18124. In the aforementioned assays, this compound has the following activity:

| | |
|---|---|
| human NK-1 receptor binding: | IC50 = 0.25 nM |
| guinea-pig vocalisation: | ID50 = 0.5 mg/kg s.c. |
| gerbil foot-tapping (5 mins.): | ID50 = 0.12 mg/kg i.v. |
| gerbil foot-tapping (24 hrs.): | ID50 = 0.17 mg/kg i.v. |

It will be appreciated from the foregoing description that an advantage of the combinations of the present invention is the oral bioavailability of the NK-1 receptor antagonists of use in such combinations. Pharmacokinetic analysis to determine the oral bioavailability of the NK-1 receptor antagonists may be effected simply by measuring the ability of the NK-1 receptor antagonist to inhibit NK-1 receptor agonist-induced foot-tapping in the gerbil following oral administration of the NK-1 receptor antagonist. Compounds with an ID₅₀≦30mg/kg p.o., and preferably ID₅₀≦10mg/kg p.o., following administration 1 hour prior to central NK-1 receptor agonist challenge are considered to be orally active according to the present invention.

### EXAMPLE 6

Alpha-2 Adrenoreceptor Activity

The alpha-2 selectivity of a compound may be determined by measuring receptor binding affinities and in vitro functional potencies in a variety of tissues known to possess alpha-2 and/or alpha-1 receptors. (See, e.g., The Alpha-2 Adrenergic Receptors, L. E. Limbird, ed., Humana Press, Clifton, N.J.) The following in vivo assays are typically conducted in rodents or other species. Central nervous system activity is determined by measuring locomotor activity as an index of sedation. (See, e.g., Spyraki, C. & H. Fibiger, “Clonidine-induced Sedation in Rats: Evidence for Mediation by Postsynaptic Alpha-2 Adrenoreceptors”, Journal of Neural Transmission, Vol. 54 (1982), pp. 153-163).

### EXAMPLE 7

Ferret Emesis: Neurokinin-1 Receptor Antagonists

Individually housed male ferrets (1.0-2.5 kg) are dosed orally by gavage with test compound. Ten minutes later they are fed with approximately 100 g of tinned cat food. At 60 minutes following oral dosing, cisplatin (10 mg/kg) is given i.v. via a jugular vein catheter inserted under a brief period of halothane anaesthesia. The catheter is then removed, the jugular vein ligated and the skin incision closed. The ferrets recover rapidly from the anaesthetic and are mobile within 10-20 minutes. The animals are observed continuously during recovery from the anaesthetic and for 4 hours following the cisplatin injection. The numbers of retches and vomits occurring during the 4 hours after cisplatin administration are recorded by trained observers.

Oral bioavailability of the NK-1 receptor antagonist is determined by its ability to inhibit cisplatin-induced emesis in ferrets following oral administration. Compounds with an ID₉₀≦3 mg/kg p.o., and preferably ID₉₀≦1 mg/kg p.o., are considered to be orally active according to the present invention. Thus, for example, the NK-1 receptor antagonist 2-(R)-(1-(R)-(3,5-bis(trifluoromethyl)phenyl)-ethoxy)-3-(S)-(4-fluorophenyl)-4-(3-(5-oxo-1H,4H-1,2,4-triazolo)methyl)morpholine, mentioned above, has an ID₉₀ in the ferret emesis assay of <3 mg/kg p.o.

### EXAMPLE 8

Ferret Emesis: PDE4 Inhibitors and Neurokinin-1 Receptor Antagonists

Experiments were conducted using male, adult, fitch or albino ferrets. They were housed in a humidity and temperature controlled environment and fed on carnivore diet (standard cat food) once a day with water ad libitum. On the day of the experiment, the animals were put in individual observation cages and left to habituate for at least 30 minutes. Following the administration of the drug or of its vehicle, they were observed continuously for a period of 120 minutes during which, the number of retches (i.e. rhythmic contraction of the abdomen) and of vomiting movements (i.e. expulsion or attempt to expel solid/liquid matter from the gastrointestinal tract) were recorded in a timely manner.

Drugs were dissolved immediately before use in 100% PEG 300 or sterile water). Oral administration (p.o.) was performed using a 10 cm feeding tube and drugs were administered in a dosing volume of 1 ml kg⁻¹. Subcutaneous (s.c.) injections were performed at the base of the neck, using sterile water or PEG 300 as the vehicle. The injection volume used was 0.5-1.0 ml kg⁻¹. For intravenous (i.v.) injections, the left jugular vein was catheterized with a polyethene cannula under halothane anaesthesia. Drugs were dissolved in PEG 300 50% in sterile water and injected in a volume of 1 ml-kg⁻¹. The vein was then ligated, the incision closed and the animals observed continuously thereafter.

Intracerebroventricular (i.c.v.) administration was performed following a surgical implantation of an i.c.v. guide cannula. Under isoflurane anaesthesia, a 22 gauge stainless steel guide cannula (total length=20 mm) was lowered 9 mm below the surface of the skull, at the midline, 2 mm anterior to the cross-suture of the posterior edge of the skull. The tip of the guide cannula is then positioned 3-5 mm directly above the area postrema. The guide cannula was fixed to the skull using 2 screws and dental acrylic cement. The animals received antibiotic (Clamoxyl LA, SmithKline Beecham, Surrey, U.K.; 15 mg kg⁻¹ s.c.) and analgesic injections (Zenecarp, C-Vet Limited, Edmounds, U.K.; 5 mg kg⁻¹ s.c.) and were housed in individual cages. They were allowed at least 1 week to recover. Intracerebroventricular administration of drugs was performed by inserting an injection unit of 24-25 mm (level of the area postrema) or 27 mm (for brain tissue injection) length into the guide cannula and infusing 2 μl of the drug solution (or vehicle) over 1 minute. The drugs were dissolved in DMSO or sterile water. Histological localisation of the site of i.c.v. injection was performed at the end of the experiment. The animals were killed with an overdose of anaesthetic (pentobarbitone sodium) and 2 μl of dye (2% pontamine sky blue) was injected via the cannula. Two minutes after the injection, the animal's skull was opened and the brain, including the cerebellum and a part of the spinal cord, was removed, frozen, and sectioned using a microtome to visualize the location of the i.c.v. injection.

Anti-emetic agents ondansetron (3 mg kg⁻¹ i.p.), and neurokinin-1 receptor antagonists (3 mg kg⁻¹ i.p. or s.c.) were administered 30 minutes prior to an oral challenge with an emetic dose of selected PDE IV inhibitors. When administered i.c.v., the anti-emetic agent was given immediately before the emetic challenge. The emetic response was monitored as described earlier.

In this example, emesis induced by inhibitors of type IV cyclic nucleotide phosphodiesterase (PDE IV) was investigated in the ferret. The PDE IV inhibitors studied were: RS 14203, R-rolipram and (R)-N-{4-[1-(3-cyclopentyloxy-4-methoxyphenyl)-2-(4-pyridyl)ethyl]phenyl}N′-ethylurea, in addition to the less active enantiomers S-rolipram and CT-3405. Following oral administrations, the incidence of emesis was positively influenced by the dose and potency of PDE IV inhibitors administered. PDE IV inhibitor-induced emesis was abolished by administration of the neurokinin-1 receptor antagonist, CP-99,994. No peripheral release of substance P by PDE IV inhibitors seems to be involved in triggering the emetic reflex since a neurokinin-1 receptor antagonist which only has peripheral neurokinin-1 receptor antagonist activity, was without effect. The implication of 5-HT3 receptors in PDE IV inhibitor-induced emesis was variable. These results indicate that a neurokinin-1 receptor antagonist may be employed with a a PDE4 inhibitor to minimize the side effects of nausea and/or emesis associated with the PDE4 inhibitor.

### EXAMPLE 9

Ferret Emesis: PDE4 Inhibitors and Alpha-2 Adrenoreceptor Agonists

Ferrets were pre-treated with the alpha2-adrenoceptor antagonist, yohimbine. Following an intraperitoneal injection, yohimbine induced retching and vomiting in all ferrets treated rapidly after dosing. A similar effect was observed whether the drug was administered orally or subcutaneously. Emesis was also recorded following the adminstration of two other selective alpha2-adrenoceptor antagonists: MK-912 and MK-467 (Pettibone et al., 1987; Clineschmidt et al., 1988). The alpha₂-adrenoceptor agonist, clonidine, was administered to ferrets at doses ranging from 62.5-250 μg kg⁻¹. By itself, clonidine did not trigger emesis. However, a light sedation that appeared to be dose-related was rapidly seen following the administration. Upon challenge with an emetic dose of the PDE4 inhibitor RS 14203 (1 mg kg⁻¹, p.o.), clonidine dose-dependently decreased the number of retches and vomits induced by RS 14203 and increased the latency of onset. At the highest dose tested (250 μg kg⁻¹), five out of six animals pre-treated with clonidine showed complete protection against RS 14203-induced emesis. The animal that did express an emetic response in that particular group experienced one retching and one vomiting episode. Similarly, clonidine (250 μg kg⁻¹) also abolished emesis induced by CT-2450 (30 mg kg⁻¹, p.o.) in all animals treated and provided complete protection in two out of three animals challenged with an emetic dose of R-rolipram (3 mg kg⁻¹, p.o.). As demonstrated in the foregoing study, emesis induced by PDE4 inhibitors was prevented by a pre-treatment with the alpha-2 adrenoreceptor agonist clonidine. These results indicate that an alpha-2 adrenoreceptor agonist may be employed with a a PDE4 inhibitor to minimize the side effects of nausea and/or emesis associated with the PDE4 inhibitor.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for the treatment or prevention of depression in a patient which comprises administering the combination of:
a PDE4 inhibitor; and
a neurokinin-1 receptor antagonist or an alpha-2 adrenoreceptor agonist, such that they give effective relief.

2. The method of claim 1 wherein the neurokinin-1 receptor antagonist is a CNS-penetrant neurokinin-1 receptor antagonist.

3. The method of claim 2 wherein the neurokinin-1 receptor antagonist is an orally active neurokinin-1 receptor antagonist.

4. The method of claim 3 wherein the neurokinin-1 receptor antagonist possesses a long duration of action.

5. A method for the treatment or prevention of depression in a patient which comprises administering the combination of:
a PDE4 inhibitor; and
a neurokinin-1 receptor antagonist or an alpha-2 adrenoreceptor agonist, wherein the neurokinin-1 receptor antagonist or the alpha-2 adrenoreceptor agonist, respectively, minimize the side effect of nausea or emesis associated with the PDE4 inhibitor.

6. The method of claim 5 wherein the neurokinin-1 receptor antagonist is a CNS-penetrant neurokinin-1 receptor antagonist.

7. The method of claim 6 wherein the neurokinin-1 receptor antagonist is an orally active neurokinin-1 receptor antagonist.

8. The method of claim 7 wherein the neurokinin-1 receptor antagonist possesses a long duration of action.

9. A method for ameliorating the symptoms attendant to employing a PDE4 inhibitor for the treatment or prevention of depression or anxiety in a patient comprising administering to the patient a neurokinin-1 receptor antagonist or an alpha-2 adrenoreceptor agonist.

10. The method of claim 10 wherein the neurokinin-1 receptor antagonist is a CNS-penetrant neurokinin-1 receptor antagonist.

11. The method of claim 11 wherein the neurokinin-1 receptor antagonist is an orally active neurokinin-1 receptor antagonist.

12. The method of claim 12 wherein the neurokinin-1 receptor antagonist possesses a long duration of action.

13. A method for reducing nausea or emesis associated with employing a PDE4 inhibitor for the treatment or prevention of depression or anxiety in a patient comprising administering to the patient a neurokinin-1 receptor antagonist or an alpha-2 adrenoreceptor agonist.

14. The method of claim 10 wherein the neurokinin-1 receptor antagonist is a CNS-penetrant neurokinin-1 receptor antagonist.

15. The method of claim 11 wherein the neurokinin-1 receptor antagonist is an orally active neurokinin-1 receptor antagonist.

16. The method of claim 12 wherein the neurokinin-1 receptor antagonist possesses a long duration of action.

17. A pharmaceutical composition comprising:
a PDE4 inhibitor; and
a neurokinin-1 receptor antagonist or an alpha-2 adrenoreceptor agonist, or both a neurokinin-1 receptor antagonist and an alpha-2 adrenoreceptor agonist, together with at least one pharmaceutically acceptable carrier or excipient.
